(19) United States
(12) Patent Application Publication
(10) Pub. No.: US 2025/0384986 A1
(43) Pub. Date: Dec. 18, 2025

(54) INTERACTIVE VOICE RESPONSE SYSTEM FOR AUTOMATED REFILL AND DELIVERY SCHEDULING FOR MEDICATION DELIVERY

(71) Applicant: Medvantx, Inc., San Diego, CA (US)

(72) Inventors: Nabil Georges Badr, San Diego, CA (US); Jeromey Lewis, San Diego, CA (US)

(21) Appl. No.: 19/239,277

(22) Filed: Jun. 16, 2025

Related U.S. Application Data
(60) Provisional application No. 63/660,715, filed on Jun. 17, 2024.

(51) Int. Cl. G16H 20/10; G06Q 10/083

(52) U.S. Cl. CPC G16H 20/10; G06Q 10/083

(57) ABSTRACT

The interactive voice response (IVR) system allows patients to request refills and schedule deliveries without having to interact with an agent. The IVR system utilizes cloud hosted call center software, business logic, a custom software workflow, and integration to a pharmacy management information system to provide a self-service solution to complete this task without the assistance of an agent. The IVR system also calculates an expected date for delivery based on information provided by the patient.

### CROSS-REFERENCE TO RELATED APPLICATIONS

This application claims priority to U.S. Provisional Application Ser. No. 63/660,715, filed Jun. 17, 2024, the entire contents of which are hereby incorporated by reference in their entirety.

### FIELD OF THE INVENTION

The present invention relates to an interactive voice response (IVR) system for automating refills and delivery scheduling of medication. The IVR system integrates a pharmacy management system with phone systems to supply the data required for the interaction between a patient and the pharmacy system.

### BACKGROUND

Patients and Healthcare Providers currently call into a pharmacy and speak with an agent to request a refill and identify potential shipping dates to ensure that their medication will arrive by the time the medication is needed. This can be a time consuming and error-prone manual process, particularly if the caller requires a different date than is available.

Patients are often subjected to long hold times, and once connected, the patient engages in a series of yes/no questions with the agent, who actively enters the appropriate data into a pharmacy management information system on their behalf. These calls represent a substantial number of the total call center calls as well as a significant percentage of overall agent time spent supporting patients and healthcare providers.

The following actions must be performed manually today by the agent after receiving the information from the patient:
Confirm patient identity.
Enter prescription number.
Determine if the patient profile is active.
Determine if their prescription is active.
Determine if the prescription has refills available.
Determine if refill due date is within the appropriate time window to refill.
Submit refill requests.
Confirm shipping address.
Confirm next available shipping date (based on complex internal business rules)
Confirm required shipping method (based on the medication's NDC and the patient's address)
Determine delivery date.
Confirm caller's availability to accept and sign for the delivery.
Submit scheduled delivery date.
Provide order confirmation number.

Therefore, a need exists for a more efficient method for identifying and scheduling medication deliveries that are also more accurate. Such a system would lead to an overall decrease in caller wait times, an increase in calls serviced, and an increase in agent availability.

### SUMMARY

The IVR system allows patients to request refills and schedule deliveries without having to interact with an agent. The IVR system utilizes cloud hosted call center software, business logic, a custom software workflow, and integration to a pharmacy management information system to provide a self-service solution to complete this task without the assistance of an agent.

This invention provides the desirable outcome of a more efficient method for identifying and scheduling medication deliveries that are also more accurate. This is expected to lead to an overall decrease in caller wait times, an increase in calls serviced, and an increase in agent availability.

### BRIEF DESCRIPTION OF THE DRAWINGS

FIG. 1 depicts a system diagram showing the components of the IVR system and the pharmacy system.

FIG. 2 depicts a flowchart showing the steps utilized for Health Insurance Portability and Accountability Act (HIPAA) verification.

FIG. 3 depicts a flowchart showing the steps utilized for prescription entry and refill request status.

FIG. 4 depicts a flowchart showing the steps utilized for refill requests and delivery scheduling.

FIG. 5 depicts a flowchart showing the steps utilized to determine the next available shipping date according to an embodiment of the invention.

FIG. 6 depicts a flowchart showing the steps utilized to allow a patient to select a shipping date.

FIG. 7 depicts a flowchart showing the steps utilized to allow a patient to schedule a delivery according to an embodiment of the invention.

In one or more implementations, not all of the depicted components in each figure may be required, and one or more implementations may include additional components not shown in a figure. Variations in the arrangement and type of the components may be made without departing from the scope of the subject disclosure. Additional components, different components, or fewer components may be utilized within the scope of the subject disclosure.

### DETAILED DESCRIPTION

The detailed description set forth below is intended as a description of various implementations and is not intended to represent the only implementations in which the subject technology may be practiced. As those skilled in the art would realize, the described implementations may be modified in various different ways, all without departing from the scope of the present disclosure. Accordingly, the drawings and description are to be regarded as illustrative in nature and not restrictive.

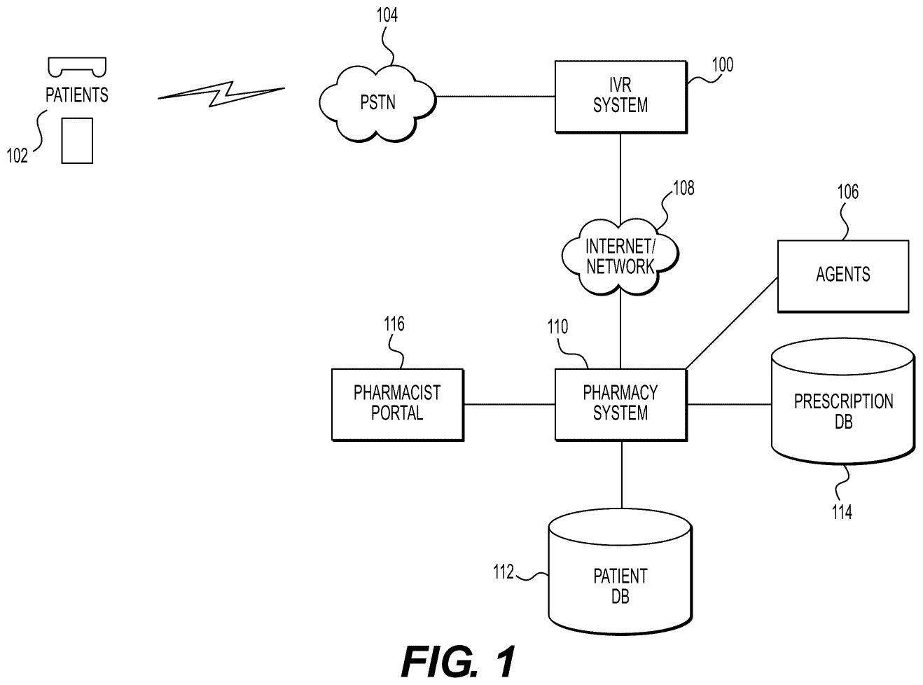

IVR system 100 allows a patient or healthcare provider to effectively submit refill requests and coordinate scheduling of medication delivery in an automated manner without the assistance of a live agent 106. As will be discussed with reference to FIGS. 1-6, IVR system 100 provides the following advantages not found in other current IVR systems or pharmacy systems:
IVR system integration with a pharmacy system for access to source of truth patient, prescription, order, and medication information.
Accurately identify active prescription and ability to refill status by entering or saying prescription number.
Securely validate patients via HIPAA approved patient information.
Efficiently submit refill request and initiate automated scheduling of medication delivery.
Confirm acceptance of delivery date, ability to sign for delivery, and receipt of order confirmation.

FIG. 1 depicts a system diagram of IVR system 100 according to an embodiment of the invention. Patients 102 can interact with IVR system 100 through any device capable of connecting to the public switched telephone network (PSTN) 104. For example, patients 102 may use a land line, cellular telephone, smartphone, tablet, computer, etc. to connect to IVR system 100.

As will be described in more detail later, IVR system 100 is able to prompt the patients 102 with questions and receive answers from the patients 102. For example, the IVR system 100 has voice recognition capabilities to translate spoken responses into the required data. The IVR system 100 can also recognize keys pressed by the patient 100 (e.g., #, *) or any combination of responses the user may provide (e.g., a spoken response followed by key presses). Patients 102 can also speak to agents 106 at any time by saying a specific phrase or pressing a specific key, such as 0. It should be obvious to one of ordinary skill in the art that any voice recognition technique may be utilized by IVR system 100.

In some embodiments, IVR system 100 is a cloud computing service hosted by one or more cloud computing providers such as Amazon or Google. The IVR system 100 can also be implemented on one or more local or remote networked servers executing the software for the IVR system 100. IVR system 100 must be capable of receiving and interpreting patient responses, coupled to the Internet 108 to bidirectionally communicate with pharmacy system 110, and coupled to PSTN 104 to receive incoming calls from patients 102.

The IVR system 100 can automatically track call reasons selected by the patient 102, such as the ability to transfer to various specific end-points such as to an external entity to order supplies, to a pharmacist to provide a verbal order, to an agent to report an adverse event, or to agents speaking other languages (e.g., Spanish). Additionally, the IVR system 100 may conducts a common NPS (Net Promoter Score) survey at the end of each call if the patient 102 has opted into taking it.

Pharmacy system 110 manages all aspects of medication and delivery scheduling for prescriptions or other medications. Once patients 102 provide their identifying information such as name, address, age, date of birth, sex, etc. to the pharmacy system 110, the identifying information is stored in a user profile in user database 112. This allows patients 102 to be identified later by IVR system 100 without having to interact with agents 106. Any prescriptions for the patient are stored in prescription database 114. A unique prescription ID is assigned to each prescription and is stored in association with the patient's user profile in patient database 112. Pharmacists, doctors, or other medical professionals can interact with pharmacist portal 116 to update patient or prescription information as needed.

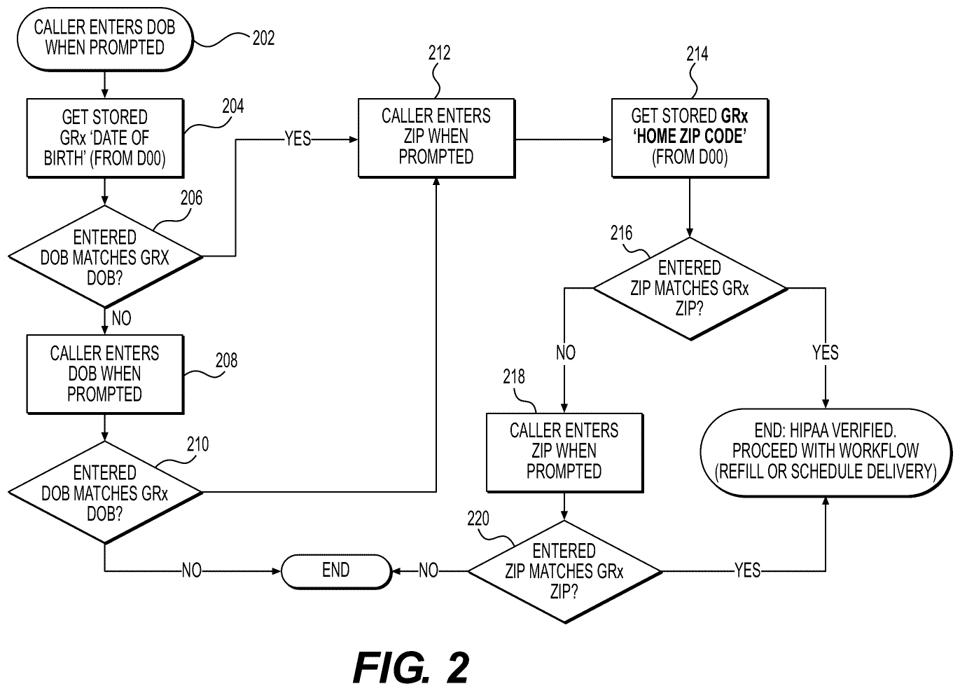

IVR system 100 and pharmacy system 110 may be operated by the same entity or different entities. It should be obvious to one of ordinary skill in the art that IVR system 100 can be modified to work in conjunction with any pharmacy system 110 capable of providing the information needed to execute the steps depicted in FIGS. 2-6. For example, the IVR system 100 can perform HIPAA verification (FIG. 2) as long as the date of birth and zip code can be retrieved form the pharmacy system 110.

FIG. 2 depicts the steps utilized by IVR system 100 for HIPAA verification prior to processing any refills or delivery scheduling for patients 102. The patient 102 is first prompted to enter their date of birth after being prompted in step 202. The IVR system 100 retrieves the date of birth information through a request to pharmacy system 100 in step 204. The entered date of birth is compared to the date of birth stored in the user profile of patient 102 in step 206.

Generally, HIPAA verification is required for any patient 102 that either does not have their prescription number, as they can utilize the alternate information workflow and provide the HIPAA verification information of their date of birth (DOB) and zip code in lieu of entering their prescription to look up their current prescription status. Additionally, if a patient 102 proceeds to submit a refill and schedule delivery, they must confirm their identity by providing HIPAA verification data of DOB and ZIP CODE prior to hearing and selecting the shipping address. If the patient 102 has entered the alternate information workflow, they have satisfied the HIPAA verification request already and will not be required to enter it again when hearing and selecting a shipping address.

If the entered date of birth does not match the stored date of birth, the patient 102 is again prompted to reenter their date of birth in step 208. If the reentered date of birth does not match the stored date of birth as decided in step 210, the call is ended by IVR system 100.

If the entered date of birth does match the stored date of birth, the patient 102 is next prompted to enter their zip code in step 212. The IVR system 100 retrieves the zip code information through a request to pharmacy system 100 in step 214. The entered zip code is compared to the date of birth stored in the user profile of patient 102 in step 216.

If the entered zip code does not match the stored zip code, the patient 102 is again prompted to reenter their zip code in step 218. If the reentered zip code does not match the stored zip code as decided in step 220, the call is ended by IVR system 100. If the reentered zip code matches the stored zip code, the patient 102 is HIPAA verified.

In the example described in FIG. 2, a combination of a date of birth and a zip code was utilized to verify patient 102. However, other combinations of identifying information may be utilized such as current age, street address, state, social security number (SSN), etc. Additionally, more than two pieces of identifying information may be required before HIPAA verification in some embodiments.

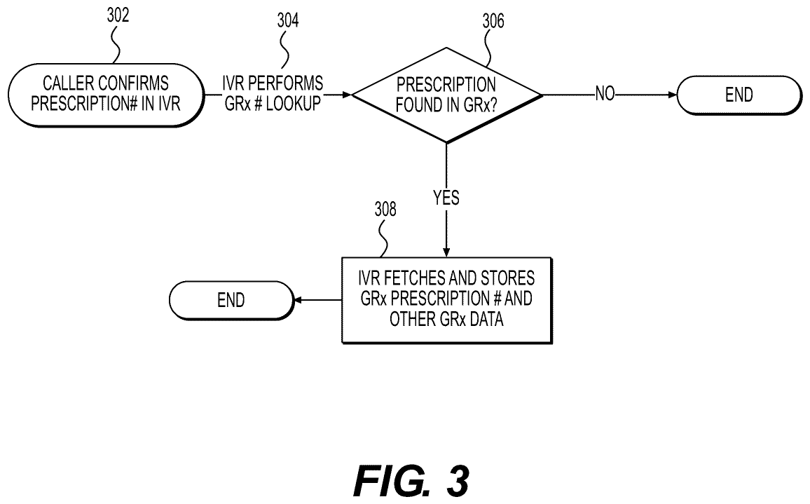

After a patient 102 has been HIPAA authenticated, they can interact with any of the features of IVR system 100 including, but not limited to, requesting refills, scheduling shipment dates, and scheduling delivery dates. FIG. 3 depicts a flowchart showing the steps utilized for prescription entry and refill request status. The patient 102 begins any request for a refill by first entering the unique prescription ID associated with the prescription in step 302. In some embodiments, the patient 102 may be required to reenter the unique prescription ID two or more times. IVR system 100 then compares the entered unique prescription ID to those stored in patient database 112 in association with the user profile in step 304. If no matching unique prescription ID is found in step 306, the call is ended by IVR system 100.

If a matching unique prescription ID is found, the associated prescription information is retrieved from prescription database 114 in step 308. Prescription information may include, but is not limited to, unique prescription ID, prescription active or inactive status, medication name, medication national drug code (NDC), number of refills remaining, total number of refills, refill due date, latest order information, date of birth, home zip code, and prescription shipping address. The latest order information may include order ID, order status, shipping date (if available), shipping method, expected by date (if available), shipping courier, and prescription tracking number(s).

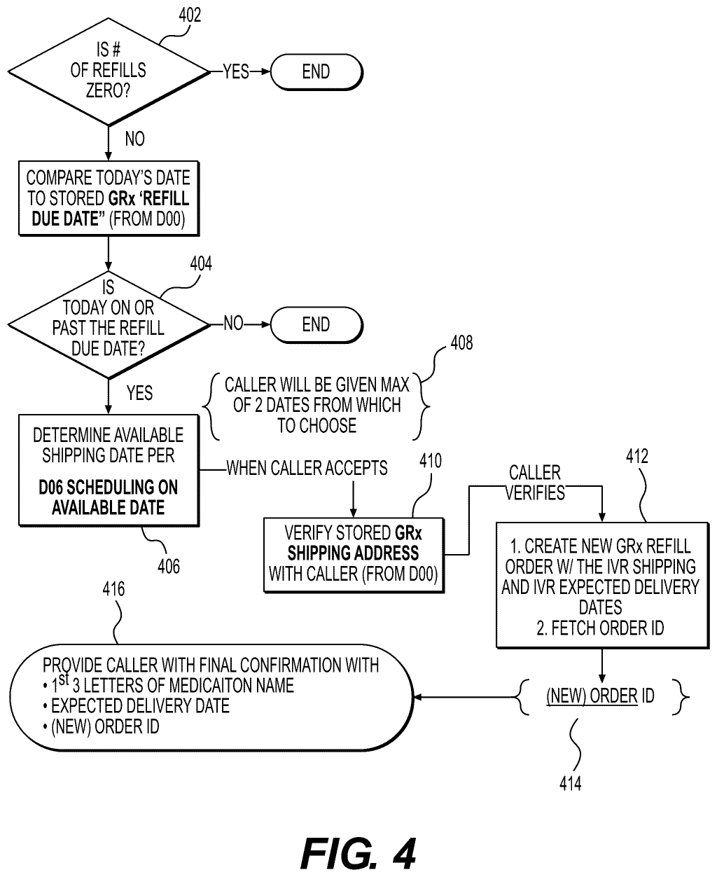

If the prescription information indicates that the prescription is active and refills are available, the patient can request a refill (e.g., by selecting a refill option or saying “Refill”). FIG. 4 depicts a flowchart showing the steps utilized for refill requests and delivery scheduling. IVR system 100 first confirms that the number of refills remaining is greater than zero in step 402. If no refills are remaining, the call is ended or the patient 102 is returned to another menu of IVR system 100. IVR system 100 next compares the current date to the refill due date in the prescription information in step 404. If the current date is not on or after the refill due date, the process is ended.

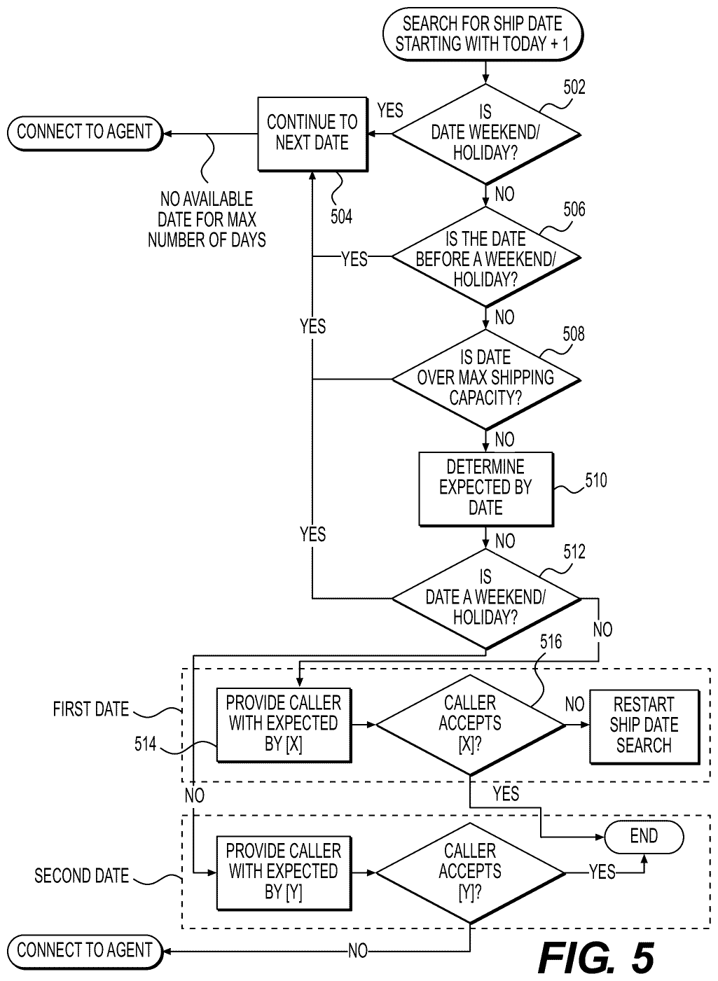

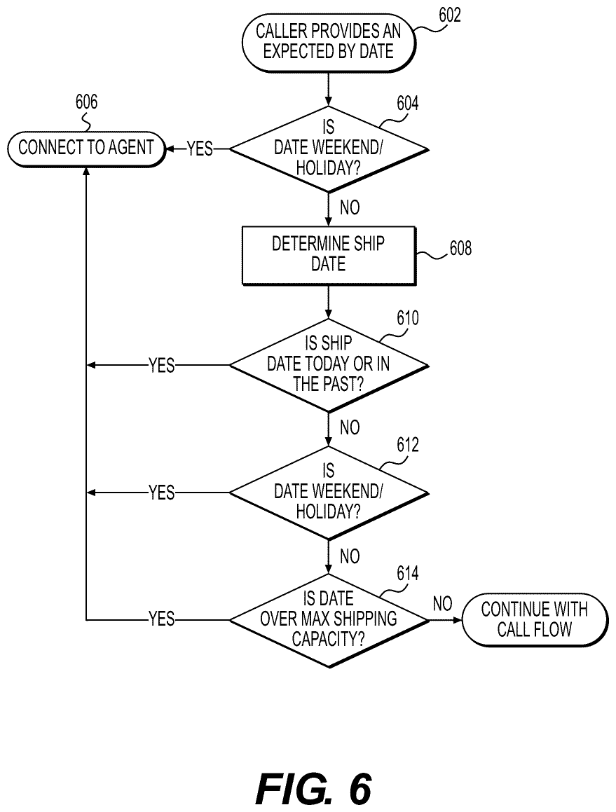

IVR system 100 determines available shipping dates utilizing the auto-scheduling process 406 discussed in more detail with reference to FIGS. 5 and 6. The patient 102 is provided with one or more available shipping dates from which to choose in step 408. After the patient 102 chooses a date, the IVR system 100 verifies the stored shipping address with the caller in step 410. A refill order is created, and an expected delivery date is calculated in step 412. The refill order is also assigned a new order ID in step 414. The patient 102 is provided with a final confirmation message in step 416 which may include the first three letters of the medication name, the expected delivery date, the new order ID, and tracking number.

FIG. 5 depicts a flowchart showing the steps utilized by IVR system 100 to determine a shipping date (e.g., presented in step 408) or to calculate an expected by date. IVR system 100 starts with the current date (to see if shipping is available day of). If the current date is a weekend or a holiday as determined in step 502, the next date is selected for review in step 504 and the process returns to step 502.

If the date is not a weekend or a holiday, IVR system 100 also determines if the date being analyzed is the date before a weekend or holiday in step 506. If the date being analyzed is the date before a weekend or holiday, the next date is selected for review in step 504. IVR system 100 next queries pharmacy system 100 to determine if the date is over maximum shipping capacity in step 508. If there is availability for shipping, an expected delivery date is calculated in step 510. If there is no availability for shipping, the next date is selected for review in step 504. The maximum shipping capacity may be different for each day selected and is adjustable. For example, the maximum shipping capacity may be reduced or increased in weeks that include a holiday.

The expected delivery date is calculated as shipping date+calculated shipping speed. If the expected delivery date is a weekend or a holiday as determined in step 512, the next shipping date is elected for review in step 504. This ensures that prescriptions are not shipped or expected on holidays or weekends.

IVR system 100 provides the expected by date to the patient 102 in step 514. If the patient 102 accepts the expected delivery date in step 516, a new prescription order is created (step 412). If the patient 102 does not accept the expected delivery date, the next date is elected for review in step 504. In some embodiments, the patient 102 is connected to an agent if two successive expected by dates are rejected.

In some embodiments, the shipping speed is a configurable value that is assigned by an operations lead within IVR system 100. The values for shipping speed may be assigned as either a ‘1’ or a ‘2.’ These values are entered per medication and when determining the estimated delivery date, the system utilizes the shipping date+the medication shipping speed to make the calculation that is shared with the caller. These values generally relate to the availability of the medication.

Instead of giving the patient 102 shipping dates to select from in step 408, the user may instead be asked to enter a desired delivery date as depicted in step 602 of FIG. 6. If the desired delivery date is a weekend or a holiday as determined in step 604, the patient 102 is connected to an agent 106 in step 606. If the desired delivery date is not a weekend or a holiday, the IVR system 100 determines a shipping date in step 608. The shipping date may be calculated by subtracting a calculated shipping speed from the desired delivery date.

IVR system 100 determines if the calculated shipping date is today or in the past in step 610. If the calculated shipping date is today or in the past, the patient 102 is connected to an agent 106 in step 606. If the calculated shipping date is not today or in the past, the IVR system 100 determines if the calculated shipping date is a weekend or a holiday in step 612.

If the calculated shipping date is a weekend or a holiday, the patient 102 is connected to an agent 106 in step 606. If the calculated shipping date is not a weekend or a holiday, the IVR system 100 determines if the calculated shipping date is over maximum shipping capacity in step 614. If the calculated shipping date is over maximum shipping capacity, the patient 102 is connected to an agent 106 in step 606. If the calculated shipping date is under the maximum shipping capacity, the user is presented with the shipping date and the call flow proceeds from step 408.

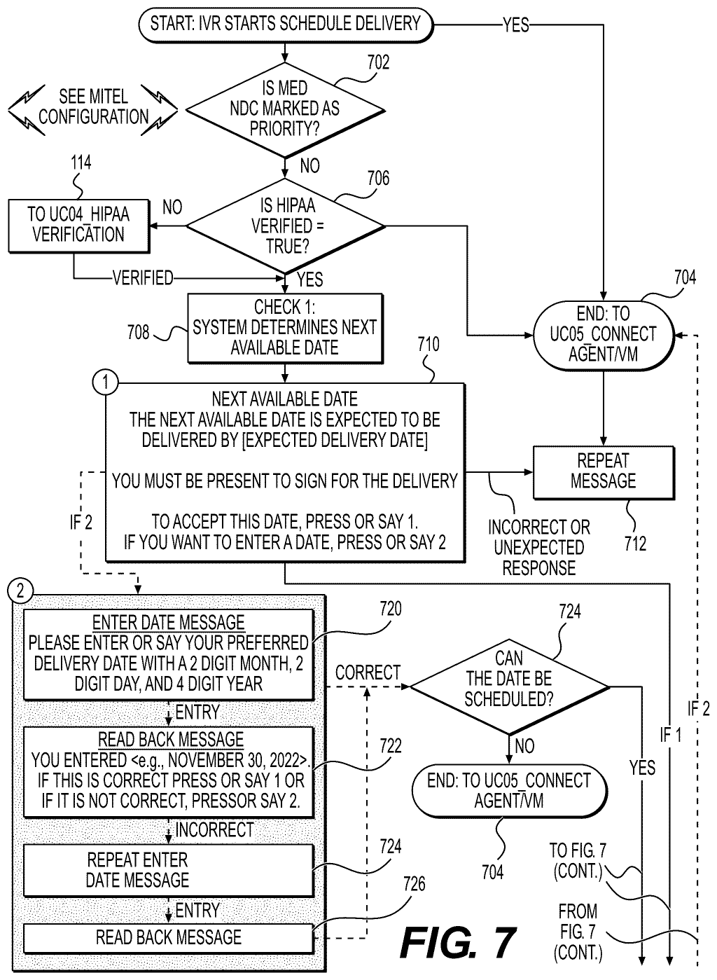

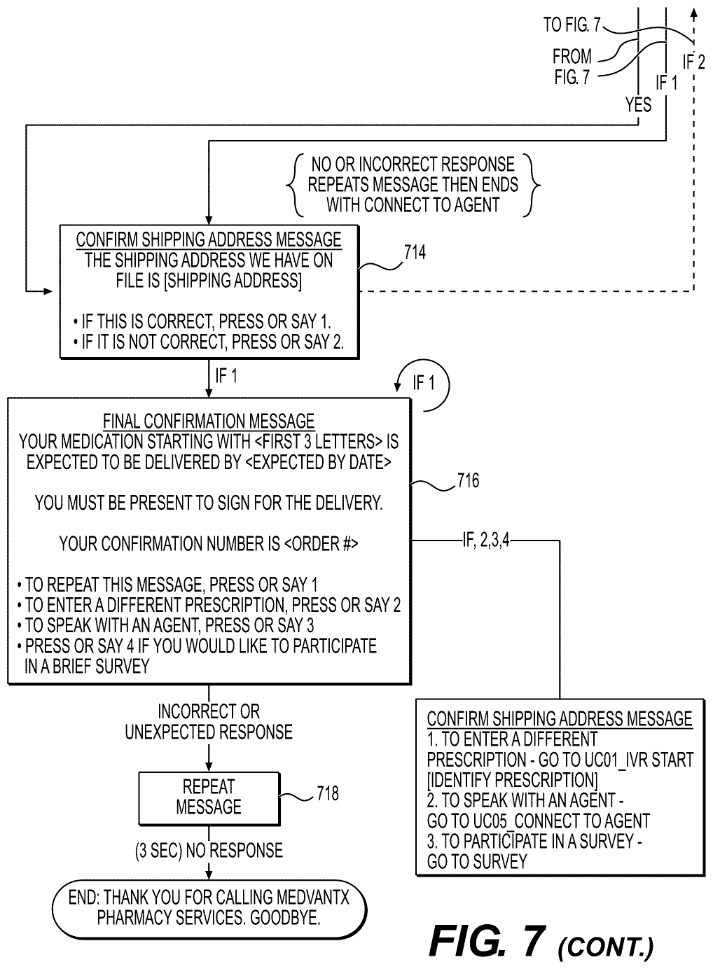

FIG. 7 depicts a flowchart showing the steps utilized to allow a patient 102 to schedule a delivery according to an embodiment of the invention. IVR system 100 first checks to determine if the medication to be delivered is national drug code (NDC) marked as a priority medication in step 702. If the medication is a priority medication, the IVR system connects the patient 102 to an agent due to the nature of the medication. If the medication is not a priority medication, the IVR system 100 can schedule delivery of the medication. Next, the patient 102 is asked to provide verification (HIPAA verification) as previously described. For example, the user may enter a DOB and zip code for verification in step 706. The information supplied by patient 702 is verified against patient database 114.

If the patient 102 is not verified, the patient 102 is directed to an agent in step 704 or the call is ended. If the patient 102 is verified, the IVR system 100 determines the next available shipping date for the patient 102 consistent with their medication to be delivered in step 708. The patient 102 is informed of the next available date through an automated prompt provided by IVR system 100 in step 710. If no response is received from patient 102, the message is repeated in step 712 a predetermined number of times (e.g., two times). If no response is received to the repeated messages, the patient 102 is connected to an agent in step 704. The patient 102 may again be required to provide HIPAA verification.

The patient 102 can accept the proposed shipping date by saying “one” or entering the number using their keypad. If the patient accepts the proposed shipping date, a confirmation message is provided to the patient 102 to confirm the shipping date in step 714. If the patient 102 confirms the shipping date, a final confirmation message is provided to the patient in step 716 with the shipping date, the medication name, the order number, and any other instructions (e.g., signature required). However, if no response is received from the patient in step 714, the patient 102 is directed to an agent in step 704. In step 716, the IVR system 100 may provide the patient 102 with additional options such as repeating the confirmation message, scheduling a delivery for another medication, speaking to an agent, or taking a survey. The final confirmation message may be repeated if no selection is received in step 718 and then the call may be ended.

Returning to step 710, the patient 102 may select that the proposed shipping date is not acceptable. The user is then asked to enter or say a preferred shipping date in step 720. The IVR system 100 will repeat the entered date to the patient 102 in step 722. If the IVR system 100 determines that the shipping date can be scheduled in step 724, the patient 102 proceeds to step 714 as previously described. If the date cannot be scheduled, the patient 102 is directed to an agent in step 704.

If the patient 102 indicates that the entered shipping date is not correct in step 722, the patient 102 is again allowed to enter a proposed shipping date in step 724. This process may repeat until the patient 102 confirms that the entered shipping date is correct in step 726. The patient then proceeds to step 724 as previously described. In the embodiment depicted in FIG. 7, the patient 102 is only provided with a single proposed shipping date by IVR system 100. For some patients 102, this is less confusing because the patient 102 only has to choose to accept the proposed shipping date (step 710) or enter their own proposed shipping date (step 720). The patient 102 must only be aware of a single shipping date at a time.

While specific embodiments of the invention have been described above, it will be appreciated that the invention may be practiced other than as described. The embodiment(s) described, and references in the specification to “one embodiment,” “an embodiment,” “an example embodiment,” “some embodiments,” etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is understood that it is within the knowledge of one skilled in the art to effect such feature, structure, or characteristic in connection with other embodiments whether or not explicitly described.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

1. A method for operating an interactive voice response (IVR) and a fulfillment system for automating refills and delivery scheduling of medication comprising:
requesting, from a user, a plurality of identification information to perform health insurance portability and accountability act (HIPAA) verification;
comparing the plurality of identification information to user information in a user database to verify the user;
if the user is verified, requesting medication information from the user related to a medication prescription;
providing the user with at least one shipment date based on the medication information and a handling capacity of the fulfillment system on the at least one shipment date;
in response to acceptance of the at least one shipment date by the user, determining an estimated delivery date for the medication prescription;
providing the user a notification with confirmation of the at least one shipment date and the estimated delivery date; and
shipping the medication prescription to the user on the at least one shipment date.

2. The method according to claim 1, wherein the plurality of identification information includes a name, a zip code, or a date of birth.

3. The method according to claim 1, wherein the at least one shipment date is not a holiday date or a weekend date.

4. The method according to claim 3, further comprising:
providing the user with another shipment date if the estimated delivery date is on a weekend or a holiday; and
updating the notification to include the another shipment date.

5. The method according to claim, wherein the at least one shipment date is further based on a medication associated with the medication prescription.

6. The method according to claim 1, wherein the estimated delivery date is based on a shipping method selected by the user and a historical shipping speed of the medication prescription.

7. The method according to claim 1, wherein the estimated delivery date is based on previous shipments to the user.

8. The method according to claim 1, wherein the user is automatically connected to a live agent if a national drug code (NDC) associated with the medication prescription is a priority medication.

9. The method according to claim 1, wherein the medication information includes a unique prescription identifier.

10. The method according to claim 9, wherein the medication prescription is shipped only if the unique prescription identifier is found in a prescription databased before the at least one shipment date.

11. The method according to claim 10, wherein the notification is updated to specify the shipping is canceled if the unique prescription identifier is not found.
